(19) United States
(12) Patent Application Publication
(10) Pub. No.: US 2011/0247616 A1
(43) Pub. Date: Oct. 13, 2011

(54) SYSTEM AND METHOD FOR DISPENSING MEDICAMENT INTO A VENTILATOR CIRCUIT

(75) Inventors: Dirk Von Hollen, Clark, NJ (US); Eric Lieberman, Scotch Plains, NJ (US)

(73) Assignee: KONINKLIJKE PHILIPS ELECTRONICS, N.V., EINDHOVEN (NL)

(21) Appl. No.: 13/132,733

(22) PCT Filed: Nov. 23, 2009
(86) PCT No.: PCT/IB2009/055298

§ 371 Date: Jun. 3, 2011

Related U.S. Application Data
(60) Provisional application No. 61/141,249, filed on Dec. 30, 2008.

(51) Int. Cl. A61M 16/10

(52) U.S. Cl. 128/203.12

(57) ABSTRACT

A drug delivery adapter is configured to facilitate the delivery of one or more medicaments to a subject being ventilated by a circuit (not shown). The circuit may be a ventilator circuit, or the like, configured to convey a pressurized flow of breathable gas to the airway of the subject. The ventilator circuit may form a flow path between a pressure generator and the airway of the subject to deliver a pressurized flow of breathable gas from the pressure generator to the airway of the subject. The drug delivery adapter, in particular, is configured to enable the delivery of one or more medicaments into the pressurized flow of breathable gas being conveyed to the subject by the circuit without compromising the integrity of the circuit through the introduction of ambient air. To facilitate this delivery, the drug delivery adapter is configured to dock one or more types of medicament dispenser.

This patent application claims the priority benefit under 35 U.S.C. §119(e) of U.S. Provisional Application No. 61/141,249 filed on Dec. 30, 2008, the contents of which are herein incorporated by reference.

The invention relates to the dispensation of medicament into a ventilator circuit.

Medicament may be dispensed into a ventilator circuit as a delivery mechanism for providing therapeutic pharmaceuticals to a subject that is being mechanically ventilated. Generally, one or more airway adaptors are provided in a ventilator circuit to enable this delivery. A typical airway adaptor includes one or more ports configured to receive a medicament dispenser for the dispensation of medicament into the ventilator circuit. Each of the ports usually is adapted to receive a single type of medicament dispenser.

One type of medicament dispenser that is accommodated by some conventional airway adaptors is a metered dose inhaler canister. These canisters are designed and tested for use with a corresponding inhaler boot. However, conventional airway adaptors are typically configured to interface with inhaler canisters, and not the inhaler boots. As such, to deliver medicament from an inhaler canister into a ventilator circuit, a caregiver or subject must dispense medicament from the inhaler canister without using the corresponding inhaler boot.

One aspect of the invention relates to a drug delivery adapter configured to be disposed within a ventilator circuit that forms a flow path between a pressure generator and the airway of a subject to deliver a pressurized flow of breathable gas from the pressure generator to the airway of the subject to mechanically ventilate the subject. In one embodiment, the adapter comprises a conduit, a drug delivery port, and a dispenser interface. The conduit forms a flow path chamber between a first opening and a second opening, and is configured such that if the conduit is disposed in a ventilator circuit, a pressurized flow of breathable gas delivered by the ventilator circuit to the airway of a subject passes through the flow path chamber from the first opening to the second opening. The drug delivery port includes a port opening, and is in fluid communication with the flow path chamber between the first opening and the second opening such that medicament introduced into the drug delivery port at the port opening is provided into the pressurized flow of breathable gas flowing through the flow path chamber. The dispenser interface is disposed at the port opening, and is configured to substantially seal the flow path chamber from ambient air entering the flow path chamber through the drug delivery port. The dispenser interface comprises a dispenser dock at which a nozzle of a medicament dispenser can be removably docked to dispense medicament into the drug delivery port without the integrity of the ventilator circuit being substantially impacted by the introduction of ambient air into the flow path chamber through the drug delivery port. The dispenser interface is formed such that the dispenser dock can removably dock a nozzle of a plurality of different types of medicament dispensers.

Another aspect of the invention relates to a drug delivery adapter configured to be disposed within a ventilator circuit that forms a flow path between a pressure generator and the airway of a subject to deliver a pressurized flow of breathable gas from the pressure generator to the airway of the subject to mechanically ventilate the subject. In one embodiment, the adapter comprises means for directing a pressurized flow of breathable gas toward the airway of a subject, the means for directing the pressurized flow being configured for disposal within a ventilator circuit; means for directing medicament into the pressurized flow of breathable gas being directed by the means for directing the pressurized flow of breathable gas; and means for removably docking a nozzle of a medicament dispenser to dispense medicament into the means for directing medicament without impacting the integrity of the ventilator circuit through the introduction of ambient air into the means for directing medicament via the means for removably docking the nozzle of the medicament dispenser, wherein the means for removably docking the nozzle of the medicament dispenser is formed to removably dock a nozzle of any of a plurality of different types of medicament dispensers.

Another aspect of the invention relates to a method of dispensing medicament into a ventilator circuit that forms a flow path between a pressure generator and the airway of a subject to deliver a pressurized flow of breathable gas from the pressure generator to the airway of the subject to mechanically ventilate the subject. In one embodiment, the method comprises directing a pressurized flow of breathable gas toward the airway of a subject through a ventilator circuit; removably docking a nozzle of a medicament dispenser to a dispenser interface that separates the ventilator circuit from ambient atmosphere, wherein the dispenser interface is formed to removably dock a nozzle of any of a plurality of different types of medicament dispensers into the ventilator circuit without substantially impacting the integrity of the ventilator circuit through the introduction of ambient air into the ventilator circuit; and receiving medicament from the medicament dispenser into the ventilator circuit via the dispenser interface, wherein the receipt of the medicament from the medicament dispenser into the ventilator circuit causes the medicament to be delivered to the airway of the subject by the pressurized flow of breathable gas being directed to the airway of the subject.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

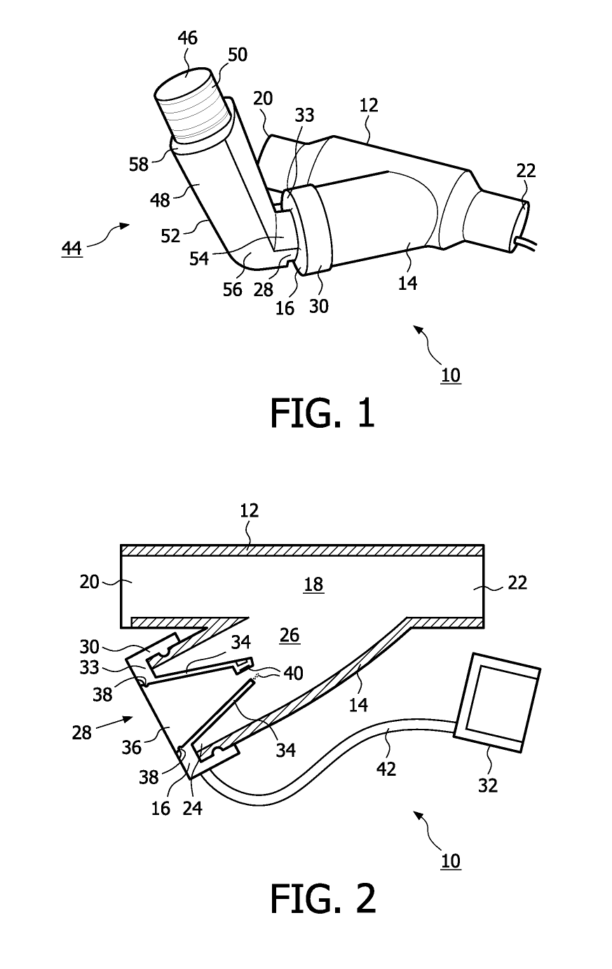

FIG. 1 illustrates a drug delivery adaptor, in accordance with one or more embodiments of the invention.

FIG. 2 illustrates a drug delivery adaptor, in accordance with one or more embodiments of the invention.

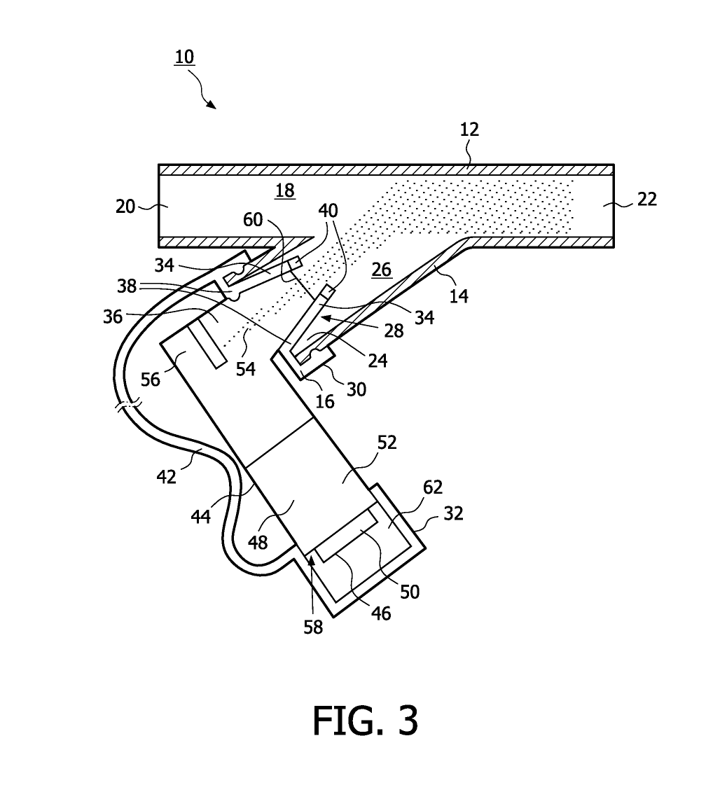

FIG. 3 illustrates a drug delivery adaptor, in accordance with one or more embodiments of the invention.

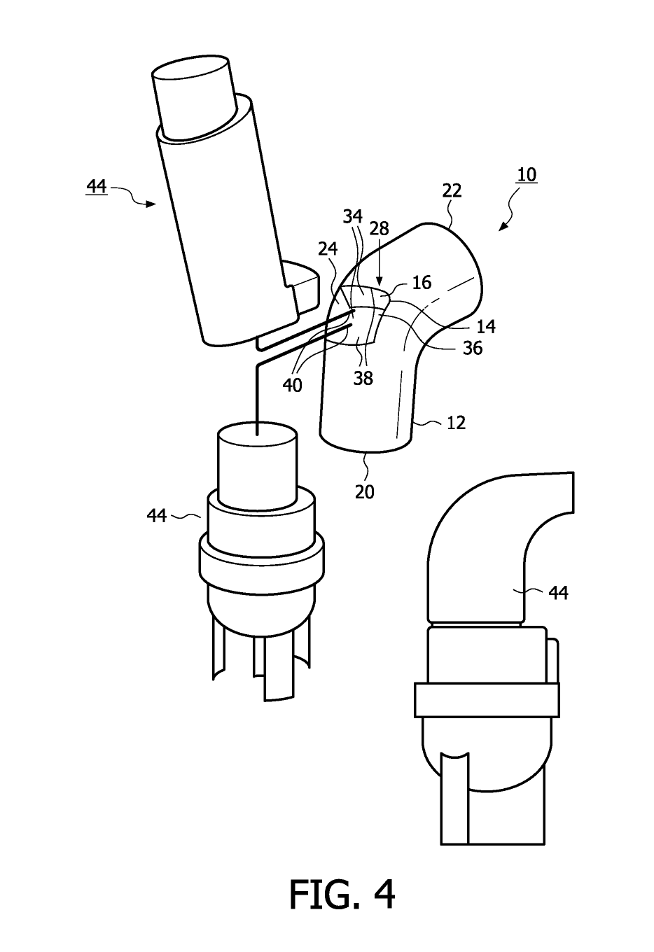

FIG. 4 illustrates a drug delivery adaptor, in accordance with one or more embodiments of the invention.

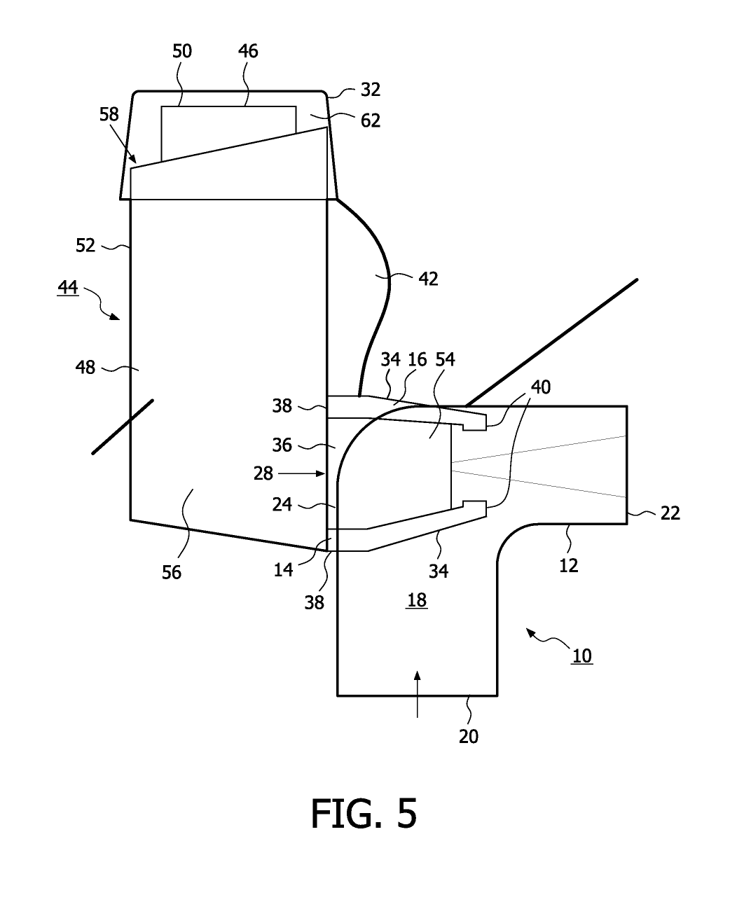

FIG. 5 illustrates a drug delivery adaptor, in accordance with one or more embodiments of the invention.

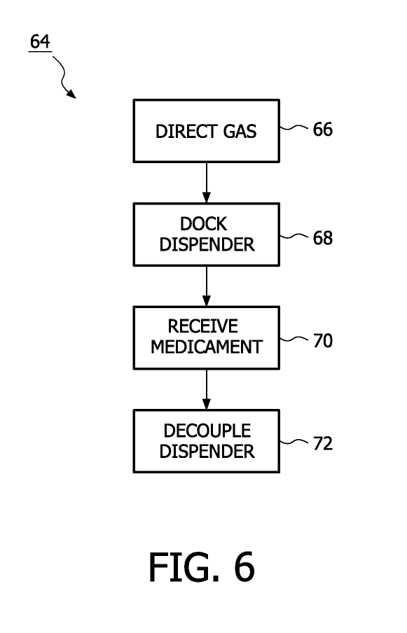

FIG. 6 illustrates a method of dispensing medicament into a ventilator circuit that forms a flow path between a pressure generator and the airway of a subject to deliver a pressurized flow of breathable gas from the pressure generator to the airway of the subject to mechanically ventilate the subject, according to one embodiment of the invention.

FIGS. 1-3 illustrate a drug delivery adapter 10 configured to facilitate the delivery of one or more medicaments to a subject being ventilated by a circuit (not shown). The circuit may be a ventilator circuit, or the like, configured to convey a pressurized flow of breathable gas to the airway of the subject. The ventilator circuit may form a flow path between a pressure generator and the airway of the subject to deliver a pressurized flow of breathable gas from the pressure generator to the airway of the subject. Drug delivery adapter 10, in particular, is configured to enable the delivery of one or more medicaments into the pressurized flow of breathable gas being conveyed to the subject by the circuit without compromising the integrity of the circuit through the introduction of ambient air. To facilitate this delivery, drug delivery adapter 10 is configured to dock one or more types of medicament dispenser. In one embodiment, drug delivery adapter 10 includes a conduit 12, a drug delivery port 14, a dispenser interface 16, and/or other components.

Referring to FIG. 2, conduit 12 forms a flow path chamber 18 between a first opening 20 and a second opening 22. Conduit 12 is configured to be disposed in a circuit (not shown), such as a ventilator circuit. When installed in the circuit, conduit 12 is configured to convey a pressurized flow of breathable gas toward a subject from first opening 20 to second opening 22 through flow path chamber 18. To ensure that the pressurized flow of breathable gas is not contaminated en route to the subject, flow path chamber 18 may be sealed, or substantially sealed from ambient atmosphere.

In the embodiment shown in FIG. 2, drug delivery port 14 includes a port opening 24 and a port chamber 26. Port opening 24 is of sufficient size to accept a medicament dispenser. During use, medicament is delivered to the interior of drug delivery adapter 10 through port opening 24. Port chamber 26 is configured to place port opening 24 in communication with flow path chamber 18 such that medicament introduced into port opening 24 passes through port chamber 26 and into the pressurized flow of breathable gas within flow path chamber 18.

Dispenser interface 16 is configured to substantially seal drug delivery port 14 from atmosphere. Dispenser interface 16 is further configured to enable a nozzle of a medicament dispenser to be removably placed into fluid communication with flow path chamber 18 without compromising the integrity of the circuit in which drug delivery adapter 10 is disposed. As such, dispenser interface 16 enables the introduction of medicament into the pressurized flow of breathable gas carried by flow path chamber 18 toward the subject without introducing significant amounts of ambient air into the pressurized flow of breathable gas. In one embodiment, dispenser interface 16 includes a dispenser dock 28, a port connector 30, and a cap 32.

Dispenser dock 28 includes a membrane 33 that forms a dock opening 36. Around dock opening 36 a plurality of flaps 34 are attached or formed integrally with membrane 33. Flaps 34 include an attached side 38 and a distal side 40. Attached side 38 attached to membrane 33 at or near dock opening 36. Distal side 40 is opposite attached side 38. Flaps 34 are formed with a dimension between attached side 38 and distal side 40 is great enough that the flaps 34 on opposite sides of dock opening 36 contact each other at distal sides 40 to substantially seal dock opening 36 in the absence of a medicament dispenser (e.g., as shown in FIG. 2) at dispenser dock 28. In particular, this dimension of flaps 34 is such that in the default position shown in FIG. 2 flaps 34 are disposed inwardly with respect to the outer housing of drug delivery adapter 10 in a duckbill configuration.

In one embodiment, dispenser dock 28 includes a mechanism that maintains the seal between distal sides 40 of flaps 34 when flaps 34 are in the default duckbill configuration shown in FIG. 2. For example, in some instances the distal sides 40 of flaps 34 are magnetized. In some instances, flaps 34 are biased toward each other. The bias of flaps 34 may be provided by the resilience of the material from which flaps 34 are formed, a spring-mechanism, and/or other biasing mechanisms. In some instances, one or more of distal sides 40 of flaps 34 has an adhesive surface that removably adheres flaps 34 together. The mechanism that maintains the seal between distal sides 40 of flaps 34 is strong enough that during inhalations by the subject interfacing with the circuit in which drug delivery adapter 10 is disposed, negative pressure within flow path chamber 10 caused by the inhalations does not cause distal sides 40 of flaps 34 to separate, thereby exposing flow path chamber 18 to contamination.

Port connector 30 is configured to connect dispenser interface 16 to drug delivery port 14. In the embodiment illustrated in FIG. 2, port connector 30 is formed from a resiliently flexible material (e.g., silicone, urethane, closed cell foam, etc.), and is stretched over port opening 24 of drug delivery port 14. In other embodiments, port connector 30 may be formed from a higher durometer material and/or may be connected to drug delivery port 14 by snap-fit, press-fit, ultrasonic welding, adhesive, and/or other techniques for attaching components. In one embodiment, dispenser dock 28 is formed integrally with drug delivery port 14. In this embodiment, dispenser interface 16 may not include port connector 30.

Cap 32 is configured, in one embodiment, to be placed in dock opening 36 externally to drug delivery adapter 10. This places cap 32 on the side of dock opening 36 opposite flaps 34 when flaps 34 are in their duckbill default position. The placement of cap 32 within dock opening 36 may further seal drug delivery port 14 to atmosphere. Prior to docking a medicament dispenser at dispenser interface 16, cap 32 may be removed from dock opening 36. In one embodiment, cap 32 is tethered to drug delivery adapter 10 via a flexible tether 42.

The sectional view of drug delivery adapter 10 shown in FIG. 3 illustrates the manner in which a medicament dispenser 44 can be docked with drug delivery adapter 10 to dispense medicament into the pressurized flow of breathable gas within flow path chamber 18. In particular, medicament dispenser 44 is a metered dose inhaler that includes a medicament canister 46 and an inhaler boot 48.

Medicament canister 46 holds the medicament, and dispenses metered doses of the medicament in aerosolized form. Medicament canister 46 includes a tank 50 and a stem (not shown in FIG. 3). Tank 50 holds medicament, along with one or more other fluids. The stem, which is attached to the end of medicament canister 46 disposed inside of inhaler boot 48 in the view shown in FIG. 3, is hollow, and dispenses medicament from tank 50 if depressed inwardly to tank 50. Within tank 50, the medicament is held with at least an aerosol propellant (e.g., a CFC-type propellant, an HFA-type propellant, etc.). When medicament canister 46 is actuated by the depression of the stem, a metered quantity of pressurized liquid (including medicament and propellant) is allowed to escape through the opening in the stem. As it escapes, the propellant in the pressurized liquid vaporizes (e.g., in 0.3 to 0.5 seconds), and the medicament that is carried by the propellant is dispersed into the surrounding gas cloud created by the vaporized propellant. The result is an aerosolized solution that includes particles of the medicament small enough to be inhaled. A solution formed with a CFC-type propellant may leave the stem at speeds between 60 and 90 miles per hour (HFA-type propellants may be a little slower).

Inhaler boot 48 holds medicament canister 46, and guides aerosolized solution from the stem of medicament canister 46 to the airway of the subject. Inhaler boot 48 includes a canister housing 52 and a mouthpiece 54. Typically, canister housing 52 and mouthpiece 54 is joined by an angled section 56. Within angled section 56, a canister seat is formed that seats medicament canister 46 inside of inhaler boot 48.

Canister housing 52 forms an opening 58 at an end opposite angled section 56, to enable medicament canister 46 to be inserted therein. Opening 58 and the conduit formed by canister housing 52 between opening 58 and angled section 56 is slightly larger than medicament canister 46 to enable air to travel from opening 58 to mouthpiece 54 between medicament canister 46 and the walls of canister housing 52 during typical use.

Mouthpiece 54 forms an opening 60 at an end opposite angled section 56.

The exterior of mouthpiece 54 is adapted to be engaged by the mouth of the subject. The conduit formed by mouthpiece 54 is configured to carry aerosolized solution from within inhaler boot 48 into the airway of the subject (e.g., through the mouth of the subject).

As can be seen in FIG. 3, in order to dock medicament dispenser 44 with dispenser interface 16 and drug delivery port 14, medicament dispenser 44 is inserted into dock opening 36 such that the nozzle of medicament dispenser 44 (e.g., mouthpiece 54) is placed in communication with flow path chamber 18. As mouthpiece 54 of medicament dispenser 44 is inserted into dock opening 36, mouthpiece 54 contacts flaps 34 and actuates flaps 34 to open. As will be appreciated from FIG. 3, the further mouthpiece 54 is inserted into dock opening 36, the further flaps 34 are actuated to accommodate the insertion.

As flaps 34 flex or pivot (e.g., by virtue of a hinge or pivotal connection at attached sides 38) to accommodate mouthpiece 54, distal sides 40 of flaps 34 remain in contact with mouthpiece 54. This contact between distal sides 40 and mouthpiece 54 forms a barrier that separates ambient air from the interior of drug delivery adapter 10 (e.g., port chamber 26, flow path chamber 18, etc.). This barrier maintains the integrity of the flow path for the pressurized flow of breathable gas through flow path chamber 18. Thus, while medicament dispenser 44 is docked at dispenser interface 16, medicament can be administered from medicament dispenser 44 into the pressurized flow of breathable gas within flow path chamber 18 without contamination caused by the introduction of gases from ambient atmosphere.

As was described above, inhaler boot 48 essentially forms a conduit from opening 58 to opening 60. During typical operation in which mouthpiece 54 is engaged by the mouth of the subject, or by a spacer or other apparatus, air at atmosphere flowing through this conduit between inhaler boot 48 and medicament canister 46 actually facilitates the delivery of medicament through mouthpiece 54. However, when medicament dispenser 44 is docked with drug delivery adapter 10 at dispenser interface 16 this airflow is a possible source of contamination to the pressurized flow of breathable gas passing through flow path chamber 18. As such, in one embodiment, cap 32 is configured to be coupled to inhaler boot 48 to effectively close opening 58. In the embodiment shown in FIG. 3, cap 32 is made from a flexible material and forms a cavity 62. Cavity 62 accommodates the portion of medicament canister 46 that extends out of canister housing 52. The flexibility of cap 32 enables a user (e.g., the subject, a caregiver, etc.) to depress medicament canister 46 to release a dose of medicament therefrom while cap 32 is still in place over opening 58. Cap 32 is secured in place by being stretched over opening 58. Other mechanisms for removably securing cap 32 to canister housing 52 are contemplated, such as snap-fit, friction-fit, and/or other mechanisms.

After dispensing medicament from medicament dispenser 44 into drug delivery adapter 10, the subject or a caregiver will remove medicament dispenser 44 from dispenser interface 16. As mouthpiece 54 is removed from dock opening 36, flaps 34 are drawn back toward the default closed position shown in FIG. 2. In particular, flaps 34 are drawn back toward the default closed position by the mechanism(s) implemented to retain flaps 34 in the closed position. For example, if distal sides 40 of flaps 34 are magnetized, the magnetic attraction between distal sides 40 will draw flaps 34 back to the default closed position. Accordingly, mouthpiece 54 can be removed from dock opening 36 with little to no ambient air entering dock opening 36 because flaps 34 continue to provide a barrier to ambient air.

One of the enhancements provided by dispenser dock 28 in enabling a medicament dispenser to be removably docked to drug delivery adapter 10 is that a variety of different types of medicament dispensers may be accommodated. The accommodation of a metered dose inhaler boot is illustrated specifically in FIG. 3, but this is not intended to be limiting. By virtue of the adaptability of flaps 34 to medicament dispenser nozzles of various shapes, dispenser dock 28 may enable other types of medicament dispensers to be removably docked with drug delivery adapter 10 to dispense medicament into flow path chamber 18 without adversely impacting the integrity of flow path chamber 18 (e.g., through the introduction of ambient air). For instance, dispenser dock 28 may accommodate a handset of a nebulizer (e.g., a jet nebulizer, an ultrasonic nebulizer, etc.). The handset may include a nozzle that is adapted to be engaged by the mouth of a subject, a nozzle configured to receive such a mouthpiece, or a handset with a nozzle that is specially designed for insertion into dock opening 36 of dispenser dock 28.

FIGS. 4 and 5 illustrate another embodiment of drug delivery adapter 10.

In particular, in the embodiment shown in FIG. 4, conduit 12 is formed as an elbow that is attached at or near second opening 22 with a patient interface appliance (e.g., a mask, a cannula, a nasal pillow, etc.) (not shown). In the embodiment illustrated in FIG. 4, drug delivery port 14 is formed at the bend in conduit 12, and dispenser interface 16 is disposed on drug delivery adapter 10 at drug delivery port 14. Providing drug delivery port 14 at the bend in conduit 12 may provide an enhanced physical accessibility to drug delivery port 14 without the bulk added to drug delivery adapter 10 by port chamber 26 in the embodiment illustrated in FIGS. 1-3. Further, since the section of a ventilator circuit that attaches to a patient interface device is typically provided as an elbow (for purposes of patient comfort), the formation of drug delivery port 14 in a bent section of conduit enables drug delivery adapter 10 to be disposed within the ventilator circuit relatively close to the airway of the subject. This may enhance the delivery of medicament to the subject through drug delivery port 14.

As can be seen in FIGS. 4 and 5, in this embodiment, dispenser interface 16 again includes dispenser dock 28 that forms a dock opening 36 lined by flaps 34. Flaps 34 shown in FIGS. 4 and 5 function in substantially the same manner as the ones illustrated in FIGS. 1-3 in enabling any of medicament dispensers 44 (illustrated in FIG. 4 as including a metered dose inhaler boot, a first nebulizer handset, and a second nebulizer handset) to be removably docked with drug delivery adapter 10 for the delivery of medicament into conduit 12. As such, flaps 34 maintain the integrity of flow path chamber 18 within conduit 12 by providing a barrier between flow path chamber 18 and ambient atmosphere that keeps the pressurized flow of breathable gas within flow path chamber 18 substantially free from ambient air. Specifically, flaps 34 operate in the manner illustrated in FIGS. 1-3 and described above to maintain the barrier as the medicament dispense is inserted into dock opening 36, while the medicament dispenser is dispensing medicament, and while the medicament dispenser is being removed from dock opening 36.

FIG. 6 illustrates a method 64 of dispensing medicament into a ventilator circuit that forms a flow path between a pressure generator and the airway of a subject to deliver a pressurized flow of breathable gas from the pressure generator to the airway of the subject to mechanically ventilate the subject. The operations of method 64 presented below are intended to be illustrative. In some embodiments, method 64 may be accomplished with one or more additional operations not described, and/or without one or more of the operations discussed. Additionally, the order in which the operations of method 64 are illustrated in FIG. 6 and described below is not intended to be limiting.

At an operation 66, a pressurized flow of breathable gas is directed toward the airway of the subject through the ventilator circuit. In one embodiment, operation 66 is performed at least in part by a conduit that is the same as or similar to conduit 12 (shown in FIGS. 1-5 and described above).

At an operation 68, a medicament dispenser is docked to a dispenser interface that separates the ventilator circuit from ambient atmosphere. The dispenser interface is formed to removably dock a nozzle of any of a plurality of different types of medicament dispensers into the ventilation circuit. In one embodiment, operation 68 is performed by a dispenser interface that is the same as or similar to dispenser interface 16 (shown in FIGS. 1-5 and described above).

At an operation 70, medicament from the medicament dispenser is received into the ventilator circuit. In one embodiment, operation 70 is performed by a dispenser interface that is the same as or similar to dispenser interface 16 (shown in FIGS. 1-5 and described above).

At an operation 72, the medicament dispenser is decoupled from the ventilator circuit without substantially compromising the integrity of the ventilator circuit through the introduction of ambient air. In one embodiment, operation 72 is performed by a dispenser interface that is the same as or similar to dispenser interface 16 (shown in FIGS. 1-5 and described above).

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A drug delivery adapter configured to be disposed within a ventilator circuit that forms a flow path between a pressure generator and the airway of a subject to deliver a pressurized flow of breathable gas from the pressure generator to the airway of the subject to mechanically ventilate the subject, the adapter comprising:
a conduit forming a flow path chamber between a first opening and a second opening, wherein the conduit is configured such that if the conduit is disposed in a ventilator circuit, a pressurized flow of breathable gas delivered by the ventilator circuit to the airway of a subject passes through the flow path chamber from the first opening to the second opening;
a drug delivery port that includes a port opening, wherein the port opening is in fluid communication with the flow path chamber between the first opening and the second opening such that medicament introduced into the drug delivery port at the port opening is provided into the pressurized flow of breathable gas flowing through the flow path chamber; and
a dispenser interface disposed at the port opening, the dispenser interface being configured to substantially seal the flow path chamber from ambient air entering the flow path chamber through the drug delivery port, the dispenser interface comprising a dispenser dock at which a nozzle of a medicament dispenser can be removably docked to dispense medicament into the drug delivery port without the integrity of the ventilator circuit being substantially impacted by the introduction of ambient air into the flow path chamber through the drug delivery port, wherein the dispenser interface is formed such that the dispenser dock can removably dock a nozzle of a plurality of different types of medicament dispensers.

2. The drug delivery adapter of claim 1, wherein the plurality of different types of medicament dispensers that can be removably docked by the dispenser interface include a metered dose inhaler boot.

3. The drug delivery adapter of claim 1, wherein the dispenser dock of the dispenser interface comprises:
a dock opening through which a nozzle of a metered dose inhaler boot or a nozzle of a nebulizer handset can be removably inserted; and
one or more flaps that seal the dock opening in the absence of a nozzle of a medicament dispenser therein, the one or more flaps being formed to be actuated by the insertion of a nozzle of a medicament dispenser such that the nozzle of the medicament dispenser is placed in communication with the drug delivery port while a seal is formed between the one or more flaps and the exterior of the nozzle.

4. The drug delivery adapter of claim 3, wherein the dispenser dock of the dispenser interface further comprises a membrane that seals the drug delivery port from atmosphere and forms the dock opening.

5. The drug delivery adapter of claim 3, wherein at least one of the one or more flaps are magnetized such that a magnetic force draws the one or more flaps into a closed position that seals the drug delivery port from ambient atmosphere.

6. The drug delivery adapter of claim 1, further comprising a boot seal configured to be removably placed over a rear opening of a metered dose inhaler boot prior to the docking of the nozzle of the metered dose inhaler boot at the dispenser dock, the boot seal preventing gas from ambient atmosphere from entering the metered dose inhaler boot through the rear opening.

7. The drug delivery adapter of claim 6, wherein the boot seal is tethered to the drug delivery adapter.

8. A drug delivery adapter configured to be disposed within a ventilator circuit that forms a flow path between a pressure generator and the airway of a subject to deliver a pressurized flow of breathable gas from the pressure generator to the airway of the subject to mechanically ventilate the subject, the adapter comprising:
means for directing a pressurized flow of breathable gas toward the airway of a subject, the means for directing the pressurized flow being configured for disposal within a ventilator circuit;
means for directing medicament into the pressurized flow of breathable gas being directed by the means for directing the pressurized flow of breathable gas; and
means for removably docking a nozzle of a medicament dispenser to dispense medicament into the means for directing medicament without impacting the integrity of the ventilator circuit through the introduction of ambient air into the means for directing medicament via the means for removably docking the nozzle of the medicament dispenser, wherein the means for removably docking the nozzle of the medicament dispenser is formed to removably dock a nozzle of any of a plurality of different types of medicament dispensers.

9. The drug delivery adapter of claim 8, wherein the plurality of different types of medicament dispensers that can be removably docked with the means for removably docking the nozzle of the medicament dispenser include a metered dose inhaler boot.

10. The drug delivery adapter of claim 8, wherein the means for removably docking the nozzle of the medicament dispenser comprises:
a dock opening through which a nozzle of a metered dose inhaler boot or a nozzle of a nebulizer handset can be removably inserted; and
one or more flaps that seal the dock opening in the absence of a nozzle of a medicament dispenser therein, the one or more flaps being formed to be actuated by the insertion of a nozzle of a medicament dispenser such that the nozzle of the medicament dispenser is placed in communication with the drug delivery port while a seal is formed between the one or more flaps and the exterior of the nozzle.

11. The drug delivery adapter of claim 10, wherein the means for removably docking the nozzle of the medicament dispenser further comprises a membrane that seals the means for directing medicament from atmosphere and forms the dock opening.

12. The drug delivery adapter of claim 10, wherein the means for removably docking the nozzle of the medicament dispenser further comprising means for drawing the one or more flaps into a closed position that seals the drug delivery port from ambient atmosphere if the nozzle is docked with the means for removably docking the nozzle of the medicament dispenser.

13. The drug delivery adapter of claim 8, further comprising means for sealing a rear opening of a metered dose inhaler boot prior to the docking of the nozzle of the metered dose inhaler boot at the dispenser dock, thereby preventing gas from ambient atmosphere from entering the metered dose inhaler boot through the rear opening.

14. The drug delivery adapter of claim 13, wherein the means for sealing the rear opening of the metered dose inhaler boot is tethered to the drug delivery adapter.

15. A method of dispensing medicament into a ventilator circuit that forms a flow path between a pressure generator and the airway of a subject to deliver a pressurized flow of breathable gas from the pressure generator to the airway of the subject to mechanically ventilate the subject, the method comprising:
directing a pressurized flow of breathable gas toward the airway of a subject through a ventilator circuit;
removably docking a nozzle of a medicament dispenser to a dispenser interface that separates the ventilator circuit from ambient atmosphere, wherein the dispenser interface is formed to removably dock a nozzle of any of a plurality of different types of medicament dispensers into the ventilator circuit without substantially impacting the integrity of the ventilator circuit through the introduction of ambient air into the ventilator circuit; and
receiving medicament from the medicament dispenser into the ventilator circuit via the dispenser interface, wherein the receipt of the medicament from the medicament dispenser into the ventilator circuit causes the medicament to be delivered to the airway of the subject by the pressurized flow of breathable gas being directed to the airway of the subject.

16. The method of claim 15, wherein the plurality of different types of medicament dispensers that can be removably docked with the dispenser interface include a metered dose inhaler boot.

17. The method of claim 15, wherein the dispenser interface comprises:
a dock opening through which a nozzle of a metered dose inhaler boot or a nozzle of a nebulizer handset can be removably inserted; and
one or more flaps that seal the dock opening in the absence of a nozzle of a medicament dispenser therein, the one or more flaps being formed to be actuated by the insertion of a nozzle of a medicament dispenser such that the nozzle of the medicament dispenser is placed in communication with the interior of the ventilation circuit while a seal is formed between the one or more flaps and the exterior of the nozzle.

18. The method of claim 17, wherein the dispenser interface further comprises a membrane that seals the interior of the ventilation circuit from atmosphere and forms the dock opening.

19. The method of claim 17, further comprising drawing the one or more flaps of the dispenser interface into a closed position that seals the interior of the ventilation circuit from ambient atmosphere as the docked nozzle of the medicament dispenser is removed from the dispenser interface.

20. The method of claim 15, further comprising sealing a rear opening of a metered dose inhaler boot prior to the docking of the nozzle of the metered dose inhaler boot at the dispenser interface, thereby preventing gas from ambient atmosphere from entering the metered dose inhaler boot through the rear opening.

21. The method of claim 20, wherein sealing the rear opening of the metered dose inhaler boot comprises sealing the rear opening with a seal that is tethered to the dispenser interface.
